(19) United States
(12) Patent Application Publication
(10) Pub. No.: US 2007/0083116 A1
(43) Pub. Date: Apr. 12, 2007

(54) Ultrasonic diagnostic apparatus

(75) Inventor: Yoshiaki Sato, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/522,964

(22) Filed: Sep. 19, 2006

(30) Foreign Application Priority Data
Sep. 21, 2005 (JP) 2005-274749

(51) Int. Cl. A61B 8/00

(52) U.S. Cl. 600/437

(57) ABSTRACT

An ultrasonic diagnostic apparatus includes an ECG memory storing ECG signals based on motion of a heart of a living body, an ECG A/D converter, and an elasticity image generating section. Upon receiving timing signals from a CPU synchronized with two points a and b in one cycle of the ECG signal, the elasticity image generating section obtains from a cinememory two frames of sound ray data of a body part to be examined. The two frames of the sound ray data correspond to the two points a and b respectively. A strain St is calculated from the obtained sound ray data. An elasticity image quantitatively indicating stiffness of the body part is generated on the basis of the calculated strain St.

### BACKGROUND OF THE INVENTION

1. Field of the Invention

The present invention relates to an ultrasonic diagnostic apparatus having a function to generate and display elasticity images quantitatively indicating elasticity of a body part.

2. Description Related to the Prior Art

Recently, in the field of medicine, ultrasonic images are widely used for diagnosis. The ultrasonic image is obtained by emitting ultrasonic waves from an ultrasonic probe toward a body part of a patient and electrically detecting echo signals from the body part by an ultrasonic observation device connected to the ultrasonic probe via a connector.

When the body part is scanned by the ultrasonic waves, an ultrasonic tomographic image (a B-mode image) is obtained. In this case, the ultrasonic probe of a mechanical scan type or of an electronic scan type is used for the ultrasonic diagnosis. In the mechanical scan type, ultrasonic transducers transmitting and/or receiving the ultrasonic waves are mechanically rotated, swung or slid. In the electronic scan type, plural ultrasonic transducers are arranged in arrays, and an electronic switch or the like selectively drives the ultrasonic transducers.

Further, recently, elastography, that is, a method for generating and displaying elasticity images quantitatively indicating elasticity (or stiffness) of the body part is suggested (see Japanese Patent Laid-Open Publications No. 2003-250803 and No. 2005-13283). According to this method, the body part of a patient is pressurized from outside, and at that time, two frames of sound ray data (digitized echo signal data) are obtained. On the basis of the above sound ray data, a strain of the body part is calculated. The elasticity image is generated by using the calculated strain. The elastography enables early diagnoses of pathologic tissues such as cancers, and facilitates determining whether tumors are benign or malignant, which have been difficult based on the conventional B-mode images.

In using an ultrasonic diagnostic apparatus disclosed in Japanese Patent Laid-Open Publication No. 2003-250803, an operator presses a tip of the ultrasonic probe against the body surface of the patient to pressurize the body part. Accordingly, the pressure amount and/or the pressurizing speed are not uniform. As a result, the elasticity image becomes discontinuous in time which makes difficult to perform the elastography. To solve this problem, the ultrasonic diagnostic apparatus disclosed in Japanese Patent Laid-Open Publication No. 2005-13283 is provided with an automatic pressing device for automatically pressing the tip of the ultrasonic probe against the body surface to improve reproducibility of the pressing operations, so that degradation in image quality of the elasticity image is prevented.

The above publications disclose the ultrasonic apparatus using the ultrasonic probe placed against the body surface. However, recent studies suggest the elastography using an ultrasonic probe inserted into the body cavity such as a small-diameter ultrasonic probe inserted into a forceps inlet of the electronic endoscope, or the ultrasonic endoscope provided with the ultrasonic transducers and the imaging sensor (see “Utility of elastography in the diagnosis of pancreatic diseases using endoscopic ultrasonography”, Hiroki UCHIDA et al., page S105 of Jpn J Med Ultrasonics Vol.32 Supplement (2005) 78-SY017). Japanese Patent Laid-Open Publication No. 2001-224594 suggests an ultrasonic endoscope apparatus which pressurizes the body part by expanding a balloon attached to the tip of the ultrasonic endoscope at the time of obtaining the elasticity image.

However, the ultrasonic endoscope apparatus disclosed in Japanese Patent Laid-Open Publication No. 20001-224594 needs the balloon and a mechanism for expanding the balloon, resulting in increasing the parts cost. The elastography using the ultrasonic probe similar to that inserted in the body cavity disclosed in the above “Utility of elastography in the diagnosis of pancreatic diseases using endoscopic ultrasonography” has a problem that the elasticity image is unstable. An ultrasonic diagnostic apparatus which enables to obtain elasticity images with stable image quality is strongly needed.

### SUMMARY OF THE INVENTION

In view of the foregoing, an object of the present invention is to provide an ultrasonic diagnostic apparatus capable of obtaining elasticity images with stable image quality.

Another object of the present invention is to provide a low cost ultrasonic diagnostic apparatus with a simple configuration by utilizing ECG signals.

To achieve the above objects and other objects, an ultrasonic diagnostic apparatus according to the present invention includes an ultrasonic probe having ultrasonic transducers for emitting ultrasonic waves toward a body part and receiving echo waves from the body part. The ultrasonic diagnostic apparatus generates an ultrasonic image from sound ray data which is digitized equivalent to the echo waves, and displays this ultrasonic image. The ultrasonic diagnostic apparatus is provided with a vibration signal acquisition section and an elasticity image generating section. The vibration signal acquisition section acquires the vibration signals based on cyclic vibrations emanating from the living body. The elasticity image generating section obtains the sound ray data of plural frames corresponding to plural points in one cycle of the vibration signals upon receiving timing signals synchronized with the plural points, and generates an elasticity image indicating stiffness of the body part based on the plural frames of the sound ray data.

The ultrasonic diagnostic apparatus of the present invention includes a first data storage for storing a plurality of frames of the sound ray data, and a second data storage for storing plural cycles of the vibration signals. The elasticity image generating section reads from the first data storage the sound ray data of plural frames corresponding to plural points in one cycle of vibration signal stored in the second data storage to generate the elasticity image.

The ultrasonic diagnostic apparatus of the present invention further includes a first image display section for displaying a waveform of the vibration signals. The ultrasonic diagnostic apparatus further includes a first setting change section for changing positions of the plural points. Furthermore, the ultrasonic diagnostic apparatus includes a second setting change section for changing a correction amount for correcting a delay between vibrations of the body part and the cyclic vibrations which are the source of the vibration signals.

In another embodiment of the present invention, the ultrasonic diagnostic apparatus includes a second image display section for displaying a body mark illustrating a shape of the living body. The ultrasonic diagnostic apparatus includes a third setting change section for changing the body part to be examined on the body mark, and a first automatic setting change section for changing the positions of the plural points in accordance with the changed body part. In this case, it is preferable that the ultrasonic diagnostic apparatus includes a second automatic setting change section for automatically changing a correction amount for correcting a delay between the vibration of the body part and the cyclic vibrations which are the source of the vibration signals, in accordance with the body part changed by the third setting change section.

It is preferable that the vibration signals are ECG signals electrically indicating motion of a heart.

It is preferable that the elasticity image generating section calculates a strain of the body part in a depth direction from the plural frames of the sound ray data.

In another embodiment of the present invention, the ultrasonic diagnostic apparatus includes a filter for removing an ultrasonic carrier component of the sound ray data, and a resampler for resampling the filtered sound ray data. The elasticity image generating section generates the elasticity image by using sound ray data processed through the filter and the resampler.

In further another embodiment of the present invention, the ultrasonic diagnostic apparatus may include a filter for removing the ultrasonic carrier component of the sound ray data, and the elasticity image generating section generates the elasticity image by using sound ray data processed through the filter.

The ultrasonic diagnostic apparatus of the present invention includes a B-mode processing section for generating a B-mode image from the sound ray data, and a third image display section for displaying the elasticity image superimposed on at least one of R, G, and B images constituting the B-mode image. In this case, it is preferable that the third image display section displays the stiffness of the body part by a gradation of color.

It is preferable that the ultrasonic probe is inserted in the body cavity for diagnosis. Further, the ultrasonic probe is an ultrasonic endoscope having an imaging sensor for capturing an optical image of a body part in the body cavity. Furthermore, it is preferable that the ultrasonic probe is a radial scan type having plural ultrasonic transducers on an outer periphery of a cylindrical surface of the ultrasonic probe.

According to the present invention, since the ultrasonic diagnostic apparatus generates the elasticity image indicating the stiffness of the body part based on the plural frames of the sound ray data corresponding to the plural points in one cycle of the vibration signals equivalent to cyclic vibration of human body, the elasticity images with stable image quality are obtained by a low-cost and simple configuration.

### BRIEF DESCRIPTION OF THE DRAWINGS

For more complete understanding of the present invention, and the advantage thereof, reference is now made to the following descriptions taken in conjunction with the accompanying drawings, in which:

FIG. 1 is a block diagram of a schematic configuration of an ultrasonic diagnostic apparatus of the present invention;

FIG. 2 is a block diagram of a schematic configuration of a B-mode processing section;

FIG. 3 is an explanatory view of a waveform of an ECG signal;

FIG. 4 is an explanatory view schematically illustrating processing for calculating a strain of a body part, performed by an elasticity image generating section;

FIG. 5 is an explanatory view schematically illustrating processing for a cine-loop playback by the elasticity image generating section;

FIG. 6 is a plan view of a console;

FIG. 7 is an explanatory view of a display example on a monitor in elastography;

FIG. 8 is an explanatory view of another display example on the monitor in the elastography;

FIG. 9 is a block diagram of another configuration of an ultrasonic observation device; and

FIG. 10 is a block diagram of yet another configuration of an ultrasonic observation device.

### DESCRIPTION OF THE PREFERRED EMBODIMENTS

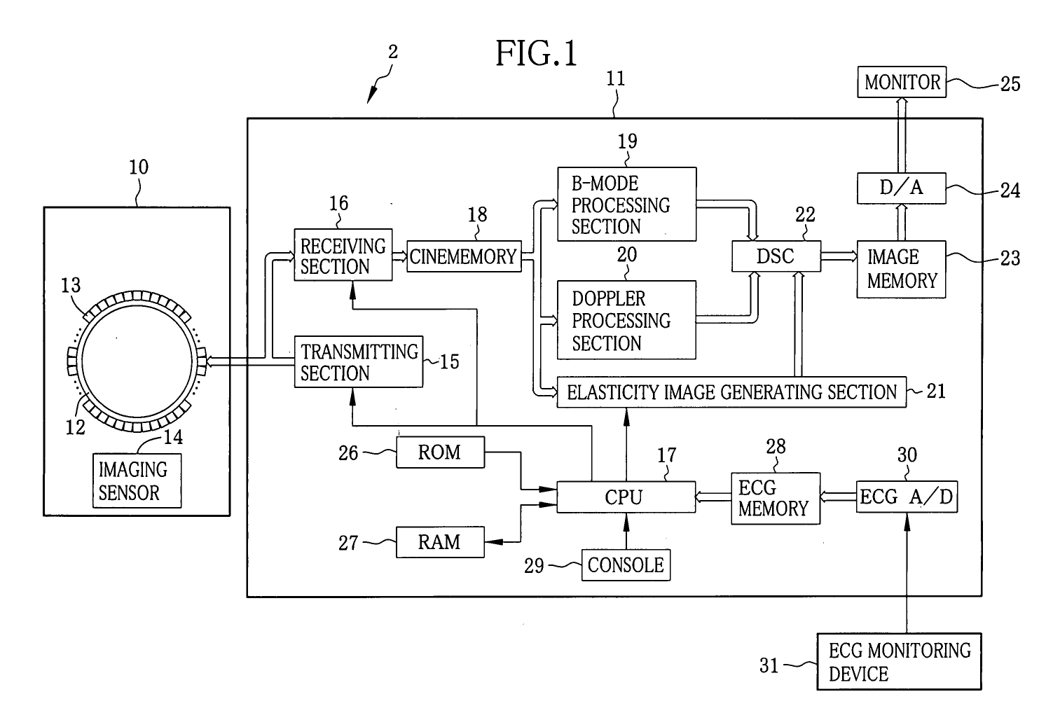

In FIG. 1, an ultrasonic diagnostic apparatus 2 is constituted of an ultrasonic endoscope 10 and an ultrasonic observation device 11 connected to the ultrasonic endoscope 10 via a connector (not shown). The ultrasonic endoscope 10 is of electronic radial scanning type in which plural ultrasonic transducers 13 are disposed on an outer periphery of a cylindrical backing member 12.

Other than the ultrasonic transducers 13, disposed on a tip of the ultrasonic endoscope 10 is an imaging sensor 14 such as a CCD which captures optical images of a body part in a body cavity. Endoscopic images generated from image signals output from the imaging sensor 14 are displayed on a monitor (not shown).

A transmitting section 15 and a receiving section 16 are connected to the ultrasonic transducers 13. To scan the ultrasound over the body part in the body cavity, a drive signal from the transmitting section 15 simultaneously drives several or several tens of the adjacent ultrasonic transducers 13 as one block. To receive echo signals from the body part, the ultrasonic transducers 13 of one block are simultaneously driven by the receiving section 16. Every time a set of the transmitting and receiving actions of the drive signal and the echo signal is done, the block to be driven is shifted by one or several adjacent ultrasonic transducers 13. Thus the ultrasonic transducers 13 to be driven are selectively switched.

The ultrasonic observation device 11 is integrally controlled by a CPU 17. The CPU 17 transmits reference pulses for determining transmission timings of the drive signals and reception timings of the echo signals to the transmitting section 15 and the receiving section 16, so as to control the operations of the transmitting section 15 and the receiving section 16.

The transmitting section 15 transmits the drive signals to the ultrasonic transducers 13, and the receiving section 16 receives the echo signals from the body part obtained by the ultrasonic transducers 13. In the receiving section 16, some of the plural echo signals are delayed for predetermined time lengths so as to phase these echo signals, and then the co-phased echo signals are combined and subjected to an A/D conversion to generate digital sound ray data.

A cinememory 18 stores a series of plural frames of the sound ray data, for instance, 100 frames, generated in the receiving section 16 at a frame rate of, for instance, 30 frames/ second. The sound ray data stored in the cinememory 18 is output to a B-mode processing section 19, a Doppler processing section 20 and an elasticity image generating section 21.

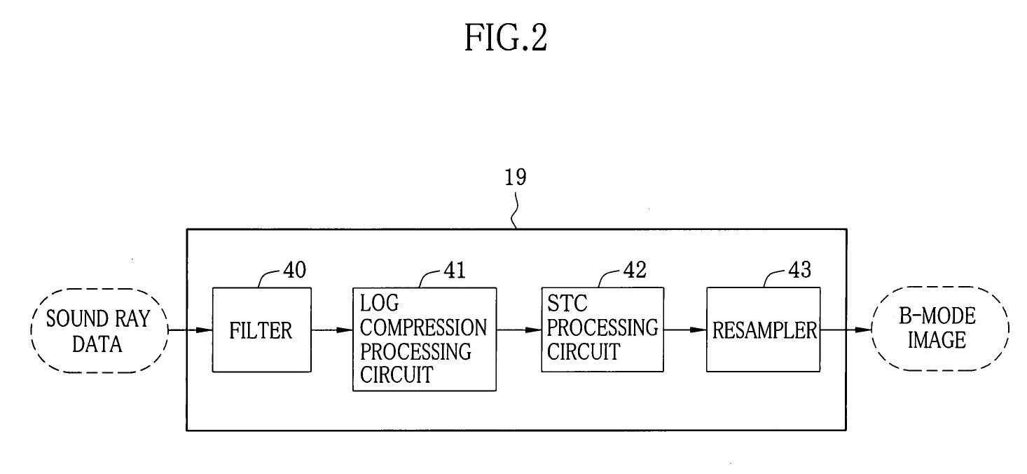

In FIG. 2, the B-mode processing section 19 includes various signal processing circuits such as a filter 40, a log compression processing circuit 41, an STC (Sensitivity Time Control) processing circuit 42, and a resampler 43. The filter 40 removes an ultrasonic carrier component of the sound ray data output from the cinememory 18. The log compression processing circuit 41 adjusts a gain and a dynamic range. The STC processing circuit 42 adjusts the amplification level with respect to a time corresponding to a propagation distance (depth) of the ultrasonic wave. The B-mode processing section 19 generates a B-mode image by applying the above signal processing to the sound ray data output from the cinememory 18 in the signal processing circuits 40 to 43.

In FIG. 1, the Doppler processing section 20 obtains blood flow information from the sound ray data output from the cinememory 18, and generates a color Doppler image by using the known CDI (color Doppler imaging) method. The elasticity image generating section 21 generates the elasticity image quantitatively indicating the stiffness of the body part from the two frames of the sound ray data.

A digital scan converter (DSC) 22 performs a raster conversion to each of the image data output from the B-mode processing section 19, the Doppler processing section 20, and the elasticity image processing section 21 to covert the image data into the signals of NTSC, a scanning method for TV. An image memory 23 stores the converted NTSC signals. A D/A converter (D/A) 24 converts the NTSC signals into the analog signals. A monitor 25 displays the converted analog signals as an image.

To the CPU 17, a ROM 26, a RAM 27, an ECG (Electrocardiogram) memory 28 and a console 29 are connected in addition to the aforementioned transmitting section 15 and the receiving section 16. The ROM 26 is, for instance, a flash memory, and stores various programs and data necessary for actuating the ultrasonic diagnostic apparatus 2. The CPU 17 reads the necessary programs and data from the ROM 26 to the RAM 27 which is a work memory and controls the operation of each section in the ultrasonic diagnostic apparatus 2.

To the ECG memory 28, an electrocardiograph 31 is connected via an A/D converter for the ECG (ECG A/D) 30. The electrocardiograph 31 electrically monitors motion of the heart of the patient, and outputs analog ECG signals in accordance with the motion of the heart. The ECG A/D 30 converts the analog ECG signals into digital ECG signals and outputs the converted digital ECG signals. The ECG memory 28 stores plural cycles, for instance, 100 cycles of the digital ECG signals.

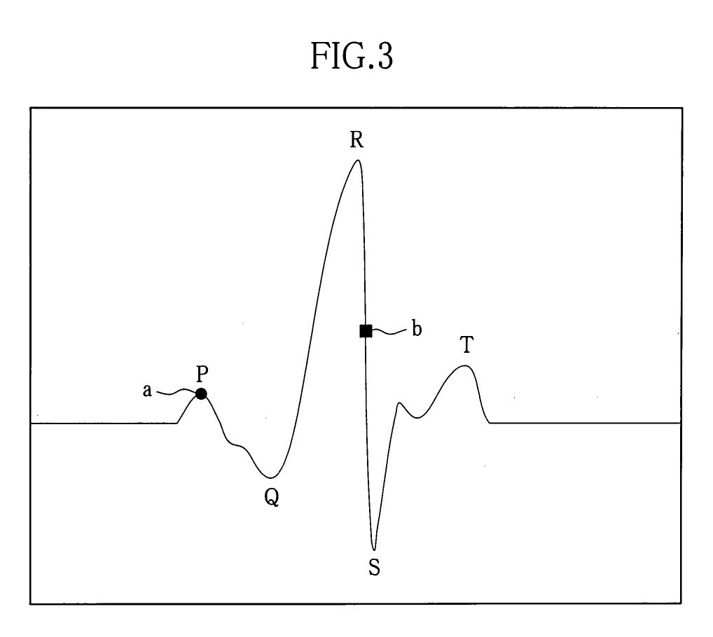

As shown in FIG. 3, each of the ECG signals stored in the ECG memory 28 is generally formed of P, Q, R, S, and T waves. The P wave represents electrical excitation of the atria. The Q, R and S waves represent electrical excitation of the ventricles. The T wave represents a repolarization process of myocardial cells in the excited ventricles. One cycle from a start of the P wave and to an end of the T wave is normally within 0.4 seconds.

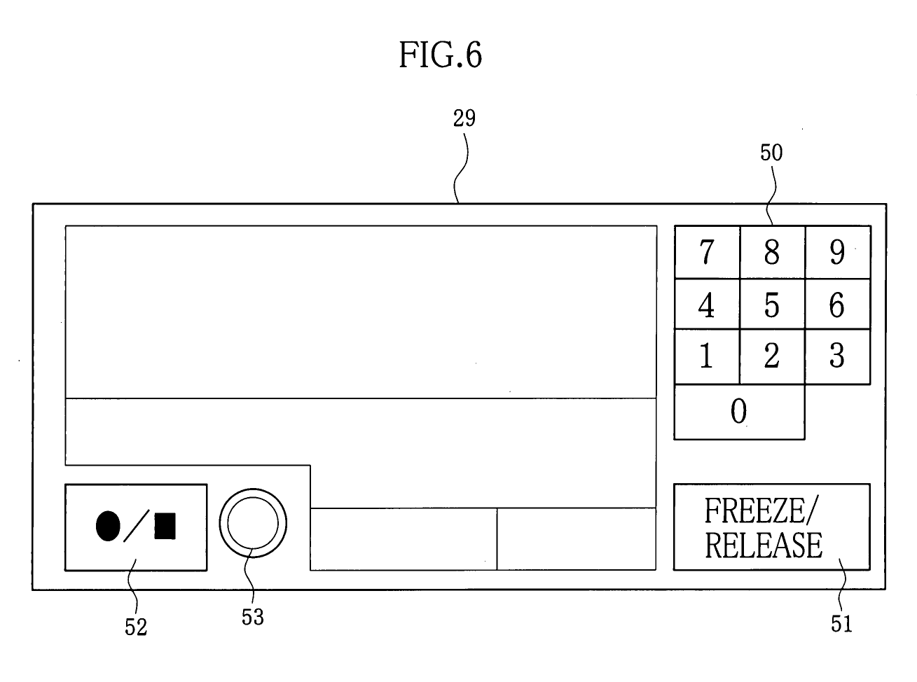

The CPU 17 generates timing signals synchronized with two points in the ECG signal stored in the ECG memory 28, for instance, a point “a” (indicated by a black circle in FIG. 3) in the proximity of a maximum point of the P wave and a point “b” (indicated by a black square) at about a midpoint of a fall of the R wave, and outputs the timing signals to the elasticity image generating section 21. The positions of the above points a and b are changeable by operating a trackball 53 provided in the console 29 (see FIG.6).

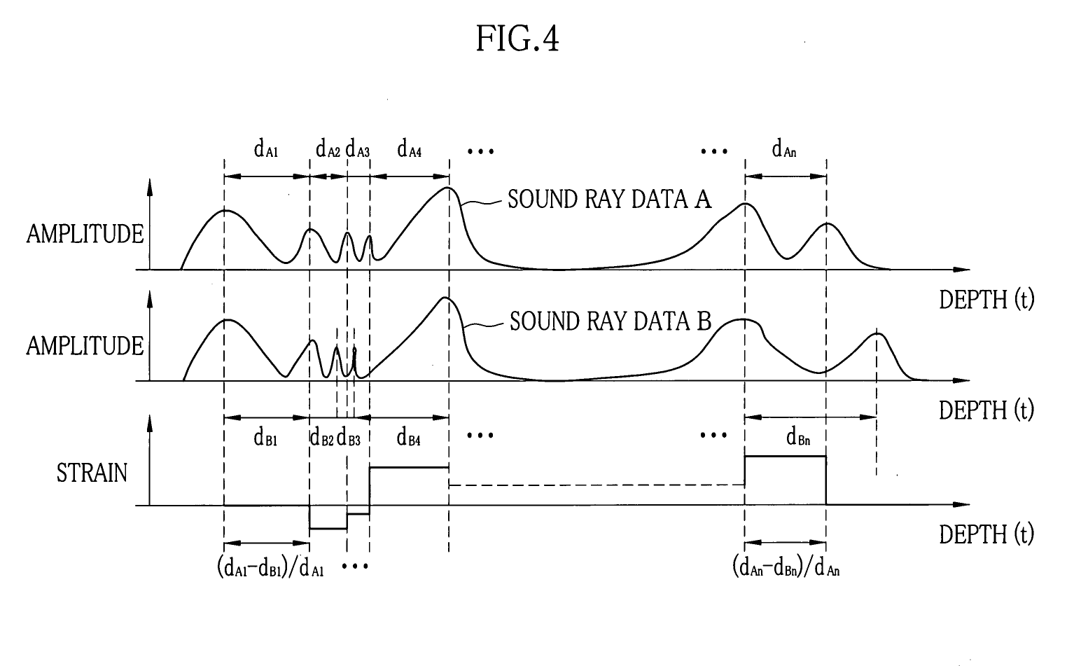

As shown in FIG. 4, the elasticity image generating section 21 obtains sound ray data A and B (two frames in total) of the body part from the cinememory 18 upon receiving the timing signals from the CPU 17. The sound ray data A and B correspond to the above two points a and b. Each of the sound ray data A and B has 1 to n+1 maximum points in the waveform, and each distance between the adjacent maximum points is calculated. The distances in the sound ray data A are indicated as d_{A1} to d_{An}, and those in the sound ray data B are indicated as d_{B1} to d_{Bn}. By using a mathematical equation below, a strain St of the body part in the depth direction is calculated.
St=(d_{Aj}−d_{Bj})/d_{Aj}(j=1to n)

In this embodiment, the strain St of the body part calculated in the elasticity image generating section 21 is used as an index indicating the stiffness of the body part.

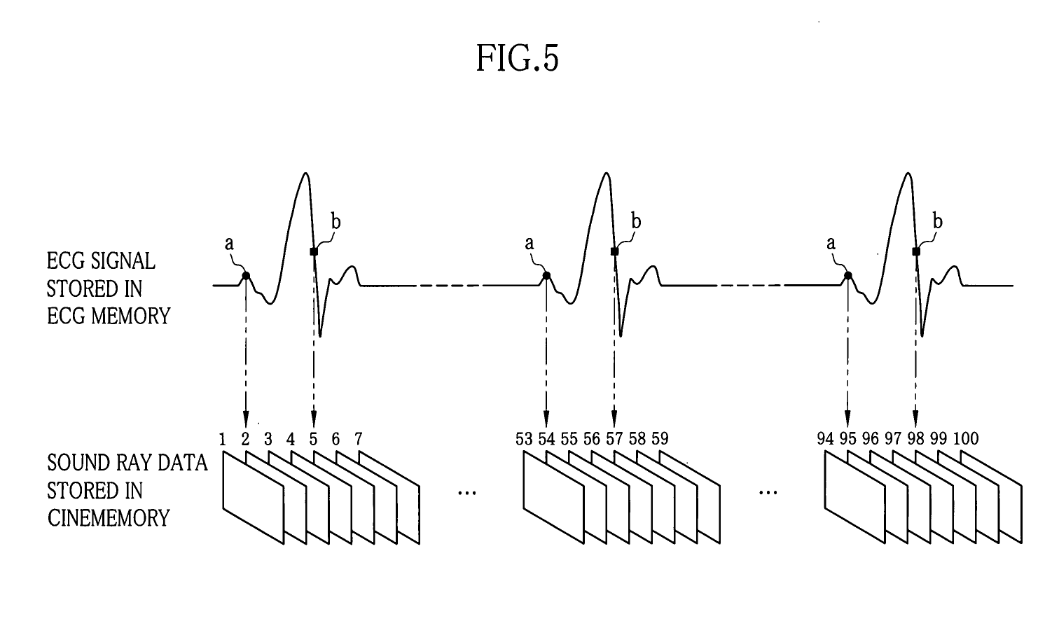

During the capture of live images after the freeze is released, the elasticity image generating section 21 reads the sound ray data from the cinememory 18 in the real time. The sound ray data is stored in the cinememory 18 in accordance with the timing signals transmitted from the CPU 17. The strain St of the body part is calculated on the basis of the read sound data. As shown in FIG. 5, it is also possible that the elasticity image generating section 21 reads from the cinememory 18 the stored sound ray data of two frames corresponding to two points in one cycle of the ECG signal stored in the ECG memory 28 (i.e. data of 2, 5, 54, 57, 95 and 98), and calculates the strain St of the body part on the basis of the read sound ray data. Thus, the above cine-loop playback enables to generate and display the elasticity images even after the diagnosis just like those generated from the live images.

As shown in FIG. 6, the console 29 is provided with a numeric keypad 50, a freeze/release switch 51, a select switch 52 and the trackball 53. The numeric keypad 50 is operated for changing the values in the settings, selecting the items and so forth. The freeze/release switch 51 is operated to pause and resume the image capturing of the ultrasonic endoscope 10. The select switch 52 is used for selecting the point “a” or “b” to be changed on the ECG signal. The trackball 53 is operated for changing the positions of the selected point “a” or “b”. The CPU 17 operates each section in the ultrasonic diagnostic apparatus 2 in response to various operation signals input from the console 29.

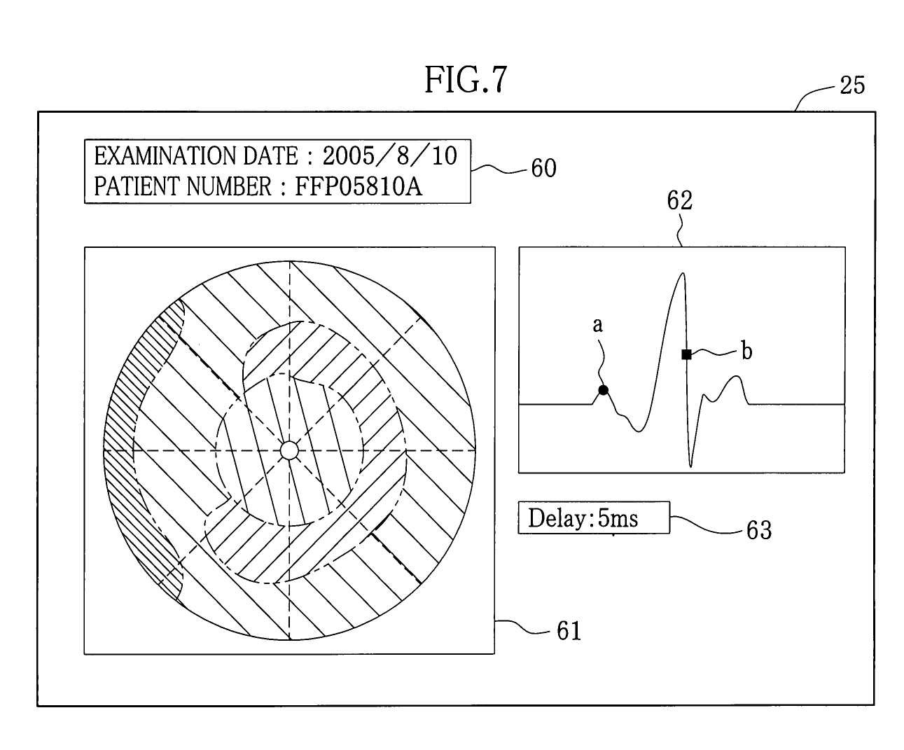

During the elastography, as shown in FIG. 7, an information window 60, an image window 61, a waveform window 62 and a delay window 63 are displayed on the monitor 25. The information window 60 displays an examination date, a patient number and so forth. The image window 61 displays the B-mode image (shown in a chain double dashed line) with which the elasticity image (indicated by hatch patterns) is synthesized. The waveform window 62 displays the waveform of the ECG signal. The delay window 63 displays a delay amount for correcting a time lag between the vibrations of the body part and the motion of the heart which is a source of the ECG signals.

The elasticity image is synthesized with an R image among R, G and B images constituting the B-mode image. The stiffness of the body part is indicated by gradation of the red color. For instance, if the density of the red color is high (meaning that the density of the hatch pattern is high), the body part is soft. If the density of the red color is low (meaning that the density of the hatch pattern is low), the body part is hard. Note that the display of the B-mode image is updated every predetermined frame rate. However, the elasticity image is updated only when the next elasticity image is generated.

A black circle and a black square representing the points a and b are synthesized with the waveform of the ECG signal. After the point to be changed is selected by the select switch 52, the black circle or the black square corresponding to the selected point is moved on the waveform by operating the trackball 53. Thereby, the position settings of the points a and b are changed. In the waveform window 62, it is possible to display the waveform of the ECG signal read from the ECG memory 28 in real time or to intermittently display a representative waveform.

It is possible to change the delay amount by inputting the appropriate value according to the body part by using the numeric keypad 50. The delay amount depends on a distance between the body part and the heart. The delay amount increases as the distance between the body part and the heart increases. On the monitor 25, the B-mode image alone, the B-mode image synthesized with the Doppler image, or the like can be displayed other than the B-mode image synthesized with the elasticity image. The display of the image is changed by operating a display change-over button (not shown) disposed in the console 29.

Next, an operation of the ultrasonic diagnostic apparatus 2 having the above configuration is described. While observing the endoscopic image obtained by the imaging sensor 14 and displayed on the monitor 25, the operator searches for a body part to be examined in the body cavity. When the tip of the ultrasonic endoscope 10 reaches the body part, the freeze is released by operating the freeze/release switch 51. At this time, under the control of the CPU 17, the drive signal is transmitted from the transmitting section 15 to a relevant block of the ultrasonic transducers 13 to drive the ultrasonic transducers 13. Thereby, the ultrasonic waves are emitted to the body part.

After the transmission of the drive signal, the transmission of the transmitting section 15 and the reception of the receiving section 16 are switched. The echo signals from the body part obtained by the ultrasonic transducers 13 are received by the receiving section 16.

In the receiving section 16, plural echo signals are phased by delaying the echo signals for predetermined time lengths, and thereafter the echo signals are combined. Then, the A/D conversion is performed to the combined echo signal to generate the digital sound ray data. The above processing is repeated throughout the last block of the ultrasonic transducers 13 while the ultrasonic transducers 13 to be driven are changed by one or several adjacent transducers 13.

After the scanning by the plural ultrasonic transducers 13 is completed, the sound ray data of one frame generated in the receiving section 16 is stored in the cinememory 18. The sound ray data stored in the cinememory 18 is output to the B-mode processing section 19, the Doppler processing section 20 and the elasticity image generating section 21.

In the B-mode processing section 19, the filter 40 removes the ultrasonic carrier component of the sound ray data output from the cinememory 18. In the log compression processing circuit 41, the gain and the dynamic range are adjusted. In the STC processing circuit 42, the sound ray data is subjected to the STC processing, and then is resampled in the resampler 43. Thereby, the B-mode image is generated.

In the Doppler processing section 20, the blood flow information is obtained from the sound ray data output from the cinememory 18, and the color Doppler image is generated by the known CDI method.

The ECG signals from the electrocardiograph 31 are converted into digital signals in the ECG A/D 30 and are sequentially stored in the ECG memory 28. The timing signals synchronized with the points a and b in the ECG signal are output from the CPU 17 to the elasticity generating section 21. The points a and b are previously determined by operating the trackball 53.

Upon receiving the timing signals from the CPU 17, the elasticity image generating section 21 reads the sound ray data of two frames corresponding to the points a and b from the cinememory 18 in real time. The strain St of the body part in the depth direction is calculated based on the above two frames of the sound ray data. Thereafter, the elasticity image is generated based on the calculated strain St.

Image data generated in each of the B-mode processing section 19, the Doppler processing section 20 and the elasticity image generating section 21 are subjected to the raster conversion in the DSC 22, and stored in the image memory 23 as the digital image data. Thereafter, the digital image data is converted into analog signals in the D/A converter 24 and displayed on the monitor 25.

In the elastography, the elasticity image synthesized with the R image of the B-mode image is displayed on the monitor 25. The stiffness of the body part is indicated by the gradation of the red color. The position of the point selected by the select switch 52 is changed by operating the trackball 53. The setting of the delay amount is changed by inputting the appropriate value by using the numeric keypad 50.

To perform the cine-loop playback, the sound ray data of two frames corresponding to two points in one cycle of the ECG signal stored in the ECG memory 28 is read from the cinememory 18 to the elasticity image generating section 21. In the elasticity image generating section 21, the elasticity image is generated based on the two frames of the sound ray data.

As described above, since the elasticity image quantitatively indicating the stiffness of the body part is generated on the basis of the sound ray data of two frames corresponding to the two points in one cycle of the ECG signal, the conventional parts and mechanisms for applying pressure onto the body part become unnecessary. Moreover, since the commonly used ultrasonic diagnostic apparatuses normally include the ECG monitoring function such as ECG memory 28 and the ECG A/D 30, it becomes possible to utilize the ultrasonic diagnostic apparatus currently used only by adding the elasticity image generating section 21 thereto.

Since plural frames of the sound ray data are stored in the cinememory 18, and plural cycles of the ECG signals are stored in the ECG memory 28 so as to generate the elasticity image by using the above data, it becomes possible to perform the cine-loop playback. Accordingly, it becomes possible to capture important images necessary for diagnosis without missing the freeze timing, which occasionally happens during the ultrasonic examination operation.

Since the positions of the two points a and b in one cycle of the ECG signal, and the delay amount are changeable, the elastography is properly performed for the intended body part. Accordingly, it becomes possible to constantly obtain the elasticity image with the stable image quality. Moreover, since the elasticity image is generated on the basis of the strain St of the body part, the time to generate the elasticity image is shortened to the extent that the elasticity image is displayed in real time. Since the elasticity image is synthesized with the R image of the B-mode image for display, a dedicated image memory for the elasticity image becomes unnecessary. As a result, the production cost is reduced.

In the body cavity, motion of the body part caused by that of the heart are large. Accordingly, the utility of the elastography is improved by applying the present invention to the ultrasonic endoscope 10 which is inserted in the body cavity as described in the above embodiment. The utility of the elastography is further improved by applying the present invention to the ultrasonic endoscope 10 of the radial scan type as described above, which enables to perform elastography in a wide display range.

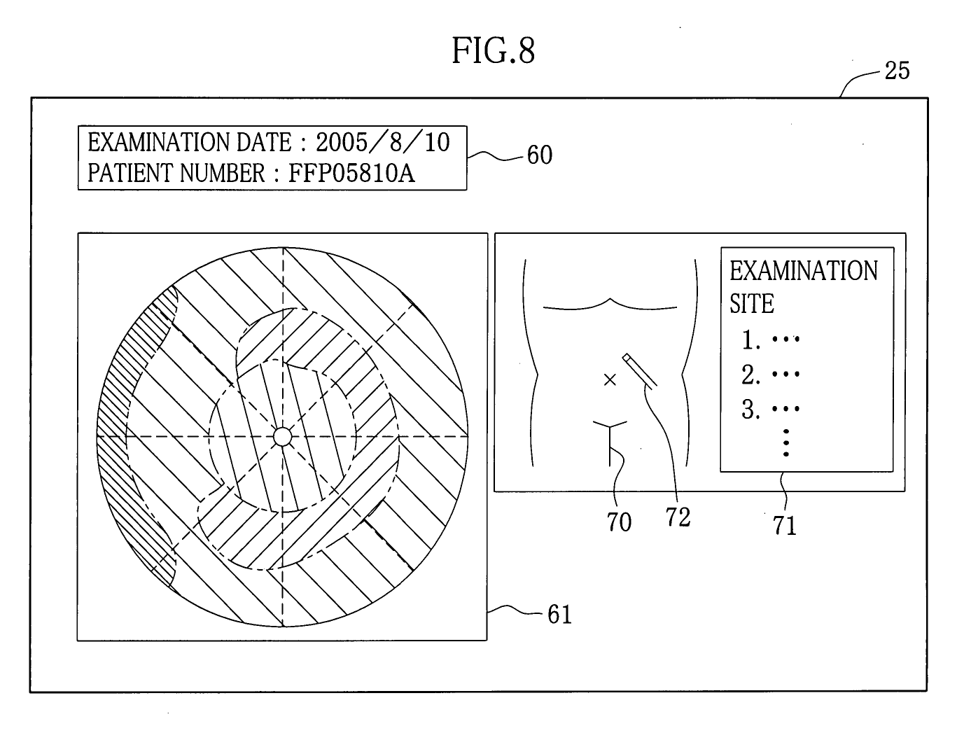

In the above embodiment, the positions of the two points a and b, and the delay amount are changeable by operating the trackball 53 and the numeric keypad 50. However, as shown in FIG. 8, it is also possible to display a body mark 70 showing an outline of the body on the monitor 25. A number corresponding to the body part to be examined is selected from an examination site list 71 having a list of numbers corresponding to different body parts by using the numeric keypad 50. Thereby, a cursor 72 on the body mark 70 is moved to the position of the selected body part, and the CPU 17 automatically changes the positions of the two points a and b, and the delay amount corresponding to the selected body part. In this case, the positions of the points a and b, and the delay amount corresponding to each body part in the examination site list 71 are previously stored in the ROM 26. At the time the body part is selected by operating the numeric keypad 50, the settings are automatically changed by reading the corresponding positions of the points a and b, and the delay amount from the ROM 26 to the CPU 17. Thus, the positions of the points a and b, and the delay amount are easily and surely changed without operating the select switch 52 or the trackball 53.

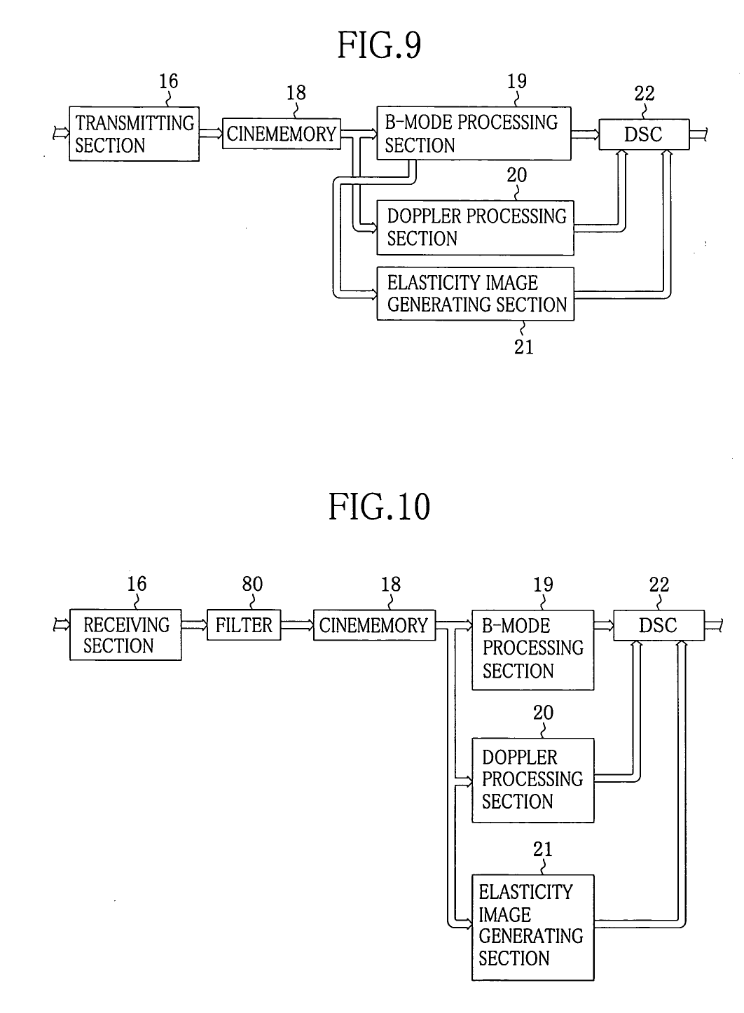

In the above embodiment, the sound ray data is directly read from the cinememory 18 to the elasticity image generating section 21. However, as shown in FIG. 9, it is possible to input the sound ray data of the B-mode processing section 19 into the elasticity image generating section 21, so that the elasticity image generating section 21 receives the sound ray data which is filtered through the filter 40 and resampled in the resampler 43. As shown in FIG. 10, it is also possible to dispose a filter 80 having similar function to the that of the filter 40 between the receiving section 16 and the cinememory 18. In both cases, the ultrasonic carrier component of the sound ray data is removed through the filter before the elasticity image generating section 21. As a result, the sound ray data with smaller data amount compared to that in the above embodiment is output to the elasticity image generating section 21, reducing the time to generate the elasticity images much further. This method is especially effective when only a few sample points (the maximum points) of the sound ray data are necessary to generate the elasticity images, for instance, in observing the changes in a submucosal layer.

In the above embodiment, the elasticity image is generated based on the sound ray data of two frames corresponding to two points in one cycle of the ECG signal. However, the number of the points is not limited to the above. It is also possible to select more than two points. In the above embodiment, the ECG signals are used as the vibration signals which are based on cyclic vibrations in the living body. However, the vibration signals are not limited to the ECG signals. It is also possible to use the vibration signals based on other spontaneous vibration of the body part, for instance, breathing, brain waves, or the like.

The present invention is not limited to the ultrasonic endoscope 10 of the radial scan type. The present invention is also effective to the ultrasonic endoscope of the convex scan type having plural ultrasonic transducers disposed in an arc shape. It is also possible to apply the present invention to the ultrasonic microprobe inserted into a forceps inlet of the electronic endoscope, and ultrasonic probe placed against the body surface.

As described so far, the present invention is not to be limited to the above embodiments, and all matter contained herein is illustrative and does not limit the scope of the present invention. Thus, obvious modifications may be made within the spirit and scope of the appended claims.

What is claimed is:

1. An ultrasonic diagnostic apparatus including an ultrasonic probe having ultrasonic transducers for emitting ultrasonic waves toward a body part in a living body and receiving echo waves from said body part, said ultrasonic diagnostic apparatus generating an ultrasonic image from sound ray data which is digitized equivalent to said echo waves and displaying said ultrasonic image, said ultrasonic diagnostic apparatus comprising:
a vibration signal acquisition section for acquiring vibration signal based on cyclic vibrations emanating from said living body; and
an elasticity image generating section for obtaining said sound ray data of plural frames corresponding to plural points in one cycle of said vibration signal when receiving timing signals synchronized with said plural points and for generating an elasticity image based on said plural frames of said sound ray data, said elasticity image indicating stiffness of said body part.

2. An ultrasonic diagnostic apparatus as claimed in claim 1, further comprising:
a first data storage for storing a plurality of frames of said sound ray data; and
a second data storage for storing plural cycles of said vibration signal;
wherein said elasticity image generating section reads from said first data storage said sound ray data of plural frames corresponding to plural points in one cycle of said vibration signal stored in said second data storage to generate said elasticity image.

3. An ultrasonic diagnostic apparatus as claimed in claim 1, further comprising:
a first image display section for displaying a waveform of said vibration signal.

4. An ultrasonic diagnostic apparatus as claimed in claim 1, further comprising:
a first setting change section for changing positions of said plural points.

5. An ultrasonic diagnostic apparatus as claimed in claim 1, further comprising:
a second setting change section for changing a correction amount for correcting a delay between said vibration of said body part and said cyclic vibration which is a source of said vibration signal.

6. An ultrasonic diagnostic apparatus as claimed in claim 1, further comprising:
a second image display section for displaying a body mark illustrating a shape of said living body;
a third setting change section for changing said body part to be examined on said body mark; and
a first automatic setting change section for changing said positions of said plural points in accordance with a change of said body part on said body mark.

7. An ultrasonic diagnostic apparatus as claimed in claim 6, further comprising:
a second automatic setting change section for automatically changing a correction amount for correcting a delay between said vibration of said body part and said cyclic vibration which is a source of said vibration signal in accordance with said change of said body part on said body mark.

8. An ultrasonic diagnostic apparatus as claimed in claim 1, wherein said vibration signal is ECG signal electrically indicating motion of a heart.

9. An ultrasonic diagnostic apparatus as claimed in claim 1, wherein said elasticity image generating section calculates a strain of said body part in a depth direction from said plural frames of said sound ray data.

10. An ultrasonic diagnostic apparatus as claimed in claim 1, further comprising:
a filter for removing an ultrasonic carrier component from said sound ray data; and
a resampler for resampling said sound ray data after said filter,
wherein said elasticity image generating section generates said elasticity image by using sound ray data processed by said filter and said resampler.

11. An ultrasonic diagnostic apparatus as claimed in claim 1, further comprising:
a filter for removing an ultrasonic carrier component from said sound ray data,
wherein said elasticity image generating section generates said elasticity image by using sound ray data processed by said filter.

12. An ultrasonic diagnostic apparatus as claimed in claim 1, further comprising:
a B-mode processing section for generating a B-mode image from said sound ray data; and
a third image display section for displaying said elasticity image superimposed on at least one of R, G, and B images constituting said B-mode image.

13. An ultrasonic diagnostic apparatus as claimed in claim 12, wherein said third image display section displays said stiffness by gradation of color.

14. An ultrasonic diagnostic apparatus as claimed in claim 1, wherein said ultrasonic probe is inserted into a body cavity for diagnosis.

15. An ultrasonic diagnostic apparatus as claimed in claim 14, wherein said ultrasonic probe is an ultrasonic endoscope having an imaging sensor for capturing an optical image of a body part in said body cavity.

16. An ultrasonic diagnostic apparatus as claimed in claim 15, wherein said ultrasonic probe is a radial scan type having plural ultrasonic transducers on an outer periphery of a cylindrical surface of said ultrasonic probe.
